(19) United States
(12) Patent Application Publication
(10) Pub. No.: US 2010/0262002 A1
(43) Pub. Date: Oct. 14, 2010

(54) Syringe Identification System

(75) Inventor: Kevin R. Martz, St. Louis, MO (US)

Correspondence Address: Mallinckrodt Inc., 675 McDonnell Boulevard, HAZELWOOD, MO, 63042, US

(73) Assignee: Mallinckrodt Inc., Hazelwood, MO (US)

(21) Appl. No.: 12/752,296

(22) Filed: Apr. 1, 2010

Related U.S. Application Data
(60) Provisional application No. 61/167,995, filed on Apr. 9, 2009.

(51) Int. Cl. A61M 5/31; G01F 11/00; A61B 6/00; G06K 7/10; B32B 38/10

(52) U.S. Cl. 600/432; 222/1; 235/454; 156/249

(57) ABSTRACT

A syringe (10′) for use with a powered injector (20) to inject a fluid into a patient that includes a length of material (1550) adapted to transmit or propagate electromagnetic energy therethrough. The length of material (1550) includes a plurality of indicators (60a-60c) positioned along the length of material. The indicators (60a-60c) are adapted to interact with at least a portion of the energy being propagated through the length of material (1550) of the syringe (10′) in a manner that is detectable. An indicator block (1500) may be disposed over at least a portion of the plurality of indicators (60a-60c). The presence (or absence) of an indicator block (1500) provides or corresponds to information about the syringe (10′) configuration. The indicator(s) (60a-60c) in combination with the indicator blocks (1500) may, for example, provide information about syringe (10′) configuration by the number and/or position thereof.

### RELATED APPLICATIONS

This application claims priority to U.S. Provisional Patent Application No. 61/167,995 filed on 9 Apr. 2009 entitled “SYRINGE IDENTIFICATION SYSTEM”, the disclosure of which is hereby incorporated by reference in its entirety.

### FIELD OF THE INVENTION

The present invention generally relates to the field of encoding and sensing of information and, more particularly, to the field of encoding information on a syringe assembly for sensing by a power injector.

### BACKGROUND

Parameters of an injection procedure are determined by a number of variables, including, for example, syringe diameter, syringe length, syringe material and fluid composition/concentration. Among the affected injection procedure parameters are fluid volume delivered, flow rate, fluid pressure, and limits of injector piston travel. In current injector systems, syringe size may be generally determined: (1) manually by action of an operator who enters the syringe size or type into the injector software; (2) automatically by means of switches on the injector head which are mechanically coupled to raised or sunken elements on the syringe; or (3) by machine reading of information associated with the syringe (e.g., barcodes, Radio Frequency Identification (RFID) tags).

As used herein, the term “syringe configuration” is used to encompass information about a particular syringe, including, but not limited to, information about the mechanical properties of a syringe (e.g., material, length and/or diameter) as well as information about the contents of the syringe (e.g., volume and/or composition). The information on syringe configuration may be used by a powered injector (alternately referred to herein as a power injector) to control the injection procedure as a function of defined syringe configuration/injection parameters. Moreover, a record of data associated with an injection procedure may be kept, for example, to satisfy accurate billing and cost information requirements under managed health care. A record may be maintained of information such as the type of syringe used, the amount of contrast medium used, the type of contrast medium used, the sterilization date, the expiration date, lot codes, the properties of the contrast media, and/or other relevant information. Such information can be recorded digitally for sharing with computerized hospital billing systems, inventory systems, control systems, and/or any other appropriate system.

### SUMMARY

The first through third aspects of the present invention are each embodied by a syringe assembly. The syringe assembly includes a body that includes a longitudinal syringe axis. The syringe assembly further includes a portion adapted to propagate energy therethrough in a direction substantially parallel to the longitudinal syringe axis. The portion includes at least two indicators disposed at predetermined positions adapted to interact with the propagating energy in a manner that is detectable. The syringe assembly further includes an indicator block disposed to block the propagation of energy from at least one of the at least two indicators to provide information about the syringe assembly configuration.

In the case of the first aspect, the syringe assembly is for use with an injector having a plurality of sensors located at different predetermined longitudinal positions on the injector. The syringe assembly of the first aspect includes a body including a wall and defining the longitudinal syringe axis. The syringe assembly further includes an mounting mechanism to enable the syringe assembly to be mounted to the injector. The syringe assembly further includes a length of material disposed along at least a portion of the wall. The length of material is adapted to propagate electromagnetic energy therethrough in a direction substantially parallel to the longitudinal syringe axis. The length of material comprises the at least two indicators. Each of the indicators is located at a different predetermined longitudinal position along the length of material and is positioned to align with a corresponding sensor when the syringe assembly is attached to the injector. Each of the indicators is adapted to interact concurrently with at least a portion of the energy being propagated through the length of material in a manner that is readily detectable by the corresponding sensor. The indicator block and the at least two indicators provide information about the syringe assembly configuration in the form of a binary code on the basis of presence or absence of electromagnetic energy from one or more of the indicators at predetermined longitudinal positions along the length of material reaching corresponding sensors. The length of material may be a portion of the wall and/or it may be a separate member positioned along at least a portion of the wall.

In the case of the second aspect, the syringe assembly includes a body defining the longitudinal syringe axis. The syringe assembly further includes a plunger movably disposed within the body. The syringe assembly further includes a length of material disposed along at least a portion of the body. The length of material is adapted to propagate light energy therethrough in a direction substantially parallel to the longitudinal syringe axis. The length of material comprises the at least two indicators. Each of the indicators is located at unique predetermined positions along the length of material. Each of the indicators is adapted to redirect at least a portion of the light energy outside of the body in a manner that is detectable. Each of the indicators is positioned at a different depth within the length of material. The indicator block is disposed to block the propagation of light energy from at least one of the at least two indicators to a corresponding sensor. The light redirected from the indicators, and not blocked by the indicator block, provides a code that provides the information about the syringe assembly configuration. The syringe assembly further includes at least one mounting flange associated with the body. The length of material may be a portion of the body and/or it may be a separate member positioned along at least a portion of the wall.

In the case of the third aspect, the syringe assembly includes a body including a wall and defining the longitudinal syringe axis. A length of the wall is adapted to propagate electromagnetic energy therethrough in a direction substantially parallel to the longitudinal syringe axis. The length of the wall includes the at least two indicators. Each of the indicators is positioned at a different depth within the wall. Each of the indicators is adapted to interact concurrently with at least a portion of the electromagnetic energy being propagated through the wall to redirect light outside of the wall in a manner that is detectable. The indicator block is disposed to block the propagation of electromagnetic energy from at least one of the at least two indicators to a corresponding sensor. The light redirected from the indicators, and not blocked by the indicator block, provides a code that provides the information about the syringe assembly configuration.

A number of feature refinements and additional features are applicable to each of the above-noted first, second, and third aspects of the present invention. These feature refinements and additional features may be used individually or in any combination in relation to each of the first, second, and third aspects. As such, each of the following features that will be discussed may be, but are not required to be, used with any other feature or combination of features of each of the first, second, and third aspects. The following discussion is applicable to each of the first, second, and third aspects, up to the start of the discussion of the fourth aspect of the present invention.

Embodiments of the syringe assembly of the first, second, and/or third aspects may be configured such that the total number of indicators may be equal to the total number of sensors in a corresponding injector to which the syringe assembly has been mounted.

Each consecutive pair of the at least two indicators may be separated by an intermediate region that includes an opaque portion of the length of material and/or wall that prevents the energy being propagated parallel to the longitudinal syringe axis from leaving the syringe assembly in a direction away from the syringe assembly (e.g., perpendicular to the longitudinal syringe axis). Each consecutive pair of the at least two indicators may be separated by an intermediate region of the length of material and/or wall that is free from a feature designed to redirect the energy away from a direction substantially parallel to the longitudinal syringe axis.

The syringe assembly may include any appropriate number of the indicators. For example, the syringe assembly may include five of the indicators. The indicator block may be in the form of a label. The indicator block may be adhesive-backed. The indicator block may include indicia related to contents of the syringe. The indicia may be human and/or machine readable. The indicator block may include at least one opaque region disposed between one of the indicators and its corresponding sensor. In an embodiment, the indicator block may include at least one transparent region disposed between one of the indicators and its corresponding sensor and at least one opaque region disposed between another one of the indicators and its corresponding sensor. The indicator block may encircle an entirety of the syringe assembly.

A fourth aspect of the present invention is embodied by a method of encoding a syringe for automated identification of the syringe. In this method, the syringe is filled with a predetermined medical fluid type and a label is selected corresponding to the predetermined medical fluid type. The selected label is then applied to the syringe such that an opaque region of the selected label is disposed over a first indicator of the syringe, while at least a second indicator of the syringe is free from having an opaque region disposed thereover. The syringe comprises a body comprising a wall and defining a longitudinal syringe axis. The first and second indicators are adapted to interact concurrently with at least a portion of energy propagated through a length of the syringe in a direction substantially parallel to the longitudinal syringe axis in a manner that is readily detectable by corresponding sensors in alignment with the first and second indicators.

A number of feature refinements and additional features are applicable to the above-noted fourth aspect of the present invention. These feature refinements and additional features may be used individually or in any combination in relation to the fourth aspect. As such, each of the following features that will be discussed may be, but are not required to be, used with any other feature or combination of features of the fourth aspect. The following discussion is applicable to the fourth aspect, up to the start of the discussion of the fifth aspect of the present invention.

The applying step of the method may further include applying the selected label such that a transparent region of the selected label is disposed over the second indicator. The applying step may further include peeling a disposable backing away from the label to expose adhesive disposed on a back side of the label, aligning one of the opaque regions with the first indicator, and contacting the back side of the label to the syringe after the aligning and peeling steps. In this regard, the label may be affixed to the syringe. The method may include shipping the syringe after the filling and applying steps such that during shipping, the syringe comprises a pre-filled syringe. In this regard, pre-filled, encoded syringes may be shipped and/or supplied to medical institutions for administration to patients.

A fifth aspect of the present invention is embodied by a syringe assembly that includes a body, a plunger, and an indicator block. The body includes a plurality of optical encoding elements adapted to transmit an optical signal. The plunger includes a plunger head movably disposed within the body. The indicator block is separately mounted on the body in position to block transmission of an optical signal from at least one of the optical encoding elements.

A number of feature refinements and additional features are applicable to the above-noted fifth aspect of the present invention. These feature refinements and additional features may be used individually or in any combination in relation to the fifth aspect. As such, each of the following features that will be discussed may be, but are not required to be, used with any other feature or combination of features of the fifth aspect. The following discussion is applicable to the fifth aspect, up to the start of the discussion of the terms “position,” “positioning” and related terms used herein.

In an embodiment, the body may include a syringe barrel. The plurality of optical encoding elements may be spaced along a longitudinal axis along which the plunger moves relative to the body.

In an arrangement, a first encoding set may correspond to first encoded information, and a second encoding set may correspond to second encoded information. The first and second encoding sets each may include at least one optical encoding element of the plurality of optical encoding elements having an optical signal that fails to be blocked by the indicator block. In an arrangement, a first encoding set may include a first combination of at least some of the plurality of optical encoding elements and may correspond with first encoded information, and a second encoding set may include a second combination of at least some of the plurality of optical encoding elements and may correspond with second encoded information. The first and second encoding sets may be different. The first encoded information may differ from the second encoded information.

The syringe assembly may include fluid in the body prior to installing the syringe assembly on an injector. The syringe assembly may include a pre-filled syringe.

The indicator block may be in the form of a label. The indicator block may be adhesive-backed. The indicator block may include indicia related to contents of the syringe assembly. The indicia may be human and/or machine readable. The indicator block may include at least one transparent region corresponding to at least one of the plurality of optical encoding elements. The indicator block may encircle an entirety of the syringe assembly.

As used herein with respect to the information provided by the indicators, the terms “position,” “positioning” and related terms refer to absolute and/or relative position. In this regard, information may be provided by the absolute position of energy emanating from one or more indicators relative to the length of material and/or wall. As used herein, the term “absolute position” refers to the position of the indicators on the length material and/or wall with respect to a reference position (e.g., a fixed position on the length of material or on a powered injector). Information may also be provided by the relative positions of a plurality of indicators with respect to each other independent of their absolute positions upon the length of material and/or wall.

As used herein in connection with electromagnetic energy transmitted and/or propagated through the length of material and/or wall, the phrase “interact with” refers generally to, for example, a transmission of the energy, a change in the direction of the energy, a change in the intensity of the energy, a change in the speed of travel of the energy and/or a change in form of the energy being propagated through the length of material. Such interactions may be readily detectable, for example, by using sensors as known in the art. For example, the indicator may be adapted to transmit the energy impinging thereupon without modification thereof, or may be adapted to transform, refract, scatter and/or absorb at least a portion of the energy. In general, the indicators may be discontinuities and/or areas having properties different from the remainder of the length of material and/or wall such that the energy impinging upon an indicator interacts differently from energy that impinges upon a portion of the length of material and/or wall not including an indicator. This different interaction of the indicator with impinging energy may be detectable. For example, an indicator may be an area of the length of material and/or wall through which energy may be transmitted outside of the length of material and/or wall, whereas the remainder of the length of material and/or wall prevents transmission of energy outside of length of material and/or wall. In the case of light energy, for example, indicators may be discontinuities such as angled surfaces formed in the length of material and/or wall that, for example, refract, reflect, scatter or absorb light energy. Indicators may also include a detection material (e.g., a fluorescent material) that may be placed in a detectable state upon impingement of the energy.

In general, a syringe assembly discussed herein may include a plurality of indicators along the length of the material and/or wall positioned at unique predetermined positions (e.g., absolute and/or relative positions). Each of the indicators may be adapted to interact with and/or to modify at least a portion of the energy being transmitted and/or propagated through the length of material in a manner that may be detectable as described above.

In an embodiment, the electromagnetic energy may be light energy and the length of material and/or wall may, for example, have a refractive index greater than the refractive index of an adjacent environment such that light energy may be internally reflected along its length. Internal reflectance may assist in efficiently propagating light energy through the length of the material and/or wall. Indicators suitable for use with light energy include, for example, angled surfaces in the syringe wall adapted to refract and/or reflect light energy outside of the syringe wall.

The length of material may, for example, be formed integrally with the syringe. In one such embodiment, the length of material may be a translucent portion of the syringe wall. Likewise, the length of material may also be separate from the syringe. The length of material may, for example, be associated with and/or attachable to the syringe. The length of material may also form part of a syringe adapter designed to adapt a syringe for use with a particular injector and/or part of a heater jacket used to warm contrast within a syringe as known in the art.

The syringe encoder may, for example, be formed integrally with, be associated with (e.g., shipped in the same container), or be attachable to a syringe assembly or a syringe adapter (designed to adapt a particular syringe for use with a powered injector).

Encoding schemes described herein provide a reliable manner of encoding information of, for example, syringe configuration. Mechanically movable mechanisms may not be required, resulting in increased reliability as compared to many prior encoding schemes. Moreover, the syringe encoders may be readily formed by disposing an appropriate indicator block over one or more indicators of the syringe. In this regard, a single syringe type may be manufactured and then the indicator may be added to identify the syringe configuration, resulting in less costly manufacture than many prior encoding mechanisms.

Furthermore, encoding systems, devices and methods described herein may be well suited for use in magnetic resonance environment. In such an environment, care should be taken to prevent failure of the encoding system or device and to prevent interference with the magnetic resonance imaging equipment. In this regard, the strong magnetic field in a magnetic resonance environment may adversely affect certain types of devices such as electromechanically activated devices. Furthermore, differences in magnetic permeability of materials within such devices and induced eddy currents therein may affect the homogeneity of the MRI magnetic field, generating image artifacts. Likewise, radio frequency energy generated by certain devices may induce unwanted artifacts upon the acquired MRI images. Such problems may be avoided in the syringe encoding systems, devices and methods described herein. For example, electromechanical and other actuators may be unnecessary as no moving elements may be required. Moreover, electromechanical energy used in the encoding systems, devices and methods may be easily selected to prevent interference with magnetic resonance equipment as well as interference from the magnetic resonance equipment. For example, light energy in the infrared, visible or ultraviolet range of the spectrum may be used. Likewise, radio frequency energy outside of frequency range of the MRI scanner may be used.

Any feature of any other various aspects of the present invention that is intended to be limited to a “singular” context or the like will be clearly set forth herein by terms such as “only,” “single,” “limited to,” or the like. Merely introducing a feature in accordance with commonly accepted antecedent basis practice does not limit the corresponding feature to the singular (e.g., indicating that a power injector includes “a syringe” alone does not mean that the power injector includes only a single syringe). Moreover, any failure to use phrases such as “at least one” also does not limit the corresponding feature to the singular (e.g., indicating that a power injector includes “a syringe” alone does not mean that the power injector includes only a single syringe). Finally, use of the phrase “at least generally” or the like in relation to a particular feature encompasses the corresponding characteristic and insubstantial variations thereof (e.g., indicating that a syringe barrel is at least generally cylindrical encompasses the syringe barrel being cylindrical).

Any “logic” that may be utilized by any of the various aspects of the present invention may be implemented in any appropriate manner, including without limitation in any appropriate software, firmware, or hardware, using one or more platforms, using one or more processors, using memory of any appropriate type, using any single computer of any appropriate type or a multiple computers of any appropriate type and interconnected in any appropriate manner, or any combination thereof. This logic may be implemented at any single location or at multiple locations that are interconnected in any appropriate manner (e.g., via any type of network).

Any power injector that may be utilized to provide a fluid discharge may be of any appropriate size, shape, configuration, and/or type. Any such power injector may utilize one or more syringe plunger drivers of any appropriate size, shape, configuration, and/or type, where each such syringe plunger driver is capable of at least bi-directional movement (e.g., a movement in a first direction for discharging fluid; a movement in a second direction for accommodating a loading and/or drawing of fluid and/or so as to return to a position for a subsequent fluid discharge operation), and where each such syringe plunger driver may interact with its corresponding syringe plunger in any appropriate manner (e.g., by mechanical contact; by an appropriate coupling (mechanical or otherwise)) so as to be able to advance the syringe plunger in at least one direction (e.g., to discharge fluid). Each syringe plunger driver may utilize one or more drive sources of any appropriate size, shape, configuration, and/or type. Multiple drive source outputs may be combined in any appropriate manner to advance a single syringe plunger at a given time. One or more drive sources may be dedicated to a single syringe plunger driver, one or more drive sources may be associated with multiple syringe plunger drivers (e.g., incorporating a transmission of sorts to change the output from one syringe plunger to another syringe plunger), or a combination thereof. Representative drive source forms include a brushed or brushless electric motor, a hydraulic motor, a pneumatic motor, a piezoelectric motor, or a stepper motor.

Any such power injector may be used for any appropriate application where the delivery of one or more medical fluids is desired, including without limitation any appropriate medical application (e.g., computed tomography or CT imaging; magnetic resonance imaging or MRI; single photon emission computed tomography or SPECT imaging; positron emission tomography or PET imaging; X-ray imaging; angiographic imaging; optical imaging; ultrasound imaging). Any such power injector may be used in conjunction with any component or combination of components, such as an appropriate imaging system (e.g., a CT scanner). For instance, information could be conveyed between any such power injector and one or more other components (e.g., scan delay information, injection start signal, injection rate).

Any appropriate number of syringes may be utilized with any such power injector in any appropriate manner (e.g., detachably; front-loaded; rear-loaded; side-loaded), any appropriate medical fluid may be discharged from a given syringe of any such power injector (e.g., contrast media, a radiopharmaceutical, saline, and any combination thereof), and any appropriate fluid may be discharged from a multiple syringe power injector configuration in any appropriate manner (e.g., sequentially, simultaneously), or any combination thereof. In one embodiment, fluid discharged from a syringe by operation of the power injector is directed into a conduit (e.g., medical tubing set), where this conduit is fluidly interconnected with the syringe in any appropriate manner and directs fluid to a desired location (e.g., to a catheter that is inserted into a patient for injection). Multiple syringes may discharge into a common conduit (e.g., for provision to a single injection site), or one syringe may discharge into one conduit (e.g., for provision to one injection site), while another syringe may discharge into a different conduit (e.g., for provision to a different injection site). In one embodiment, each syringe includes a syringe barrel and a plunger that is disposed within and movable relative to the syringe barrel. This plunger may interface with the power injector's syringe plunger drive assembly such that the syringe plunger drive assembly is able to to advance the plunger in at least one direction, and possibly in two different, opposite directions.

As used herein, the term “fluidly interconnected” refers to two or more components or entities being connected (directly or indirectly) in a manner such that fluid can flow (e.g., unidirectionally or bidirectionally) in a predetermined flow path therebetween. For example, “an injection device fluidly interconnected to a patient” describes a configuration where fluid can flow from the injection device through any interconnecting devices (e.g., tubing, connectors) and into the patient (e.g., into the vasculature of the patient).

### BRIEF DESCRIPTION OF THE FIGURES

FIG. 1 illustrates a side cross-sectional view of a prior art syringe encoding system,

FIG. 2 illustrates total internal reflectance of light within a prior art syringe wall material.

FIG. 3 illustrates a side cross-sectional view of another prior art syringe encoding system.

FIG. 4A illustrates a side cross-sectional view of a prior art syringe encoder in which an indicator scatters light to be detected by a corresponding sensor.

FIG. 4B illustrates a side cross-sectional view of a prior art syringe encoder in which an indicator absorbs light.

FIG. 4C illustrates a side cross-sectional view of a prior art syringe encoder in which an indicator acts as a lens to focus light upon a corresponding sensor.

FIG. 4D illustrates a side cross-sectional view of a prior art syringe encoder in which an indicator enters into an “excited” state detectable by a corresponding sensor when the indicator is contacted by electromagnetic energy.

FIG. 4E illustrates a side cross-sectional view of another prior art syringe encoder similar to that of FIG. 4D in which a source of electromagnetic energy is placed in generally the same plane as the sensors thereof.

FIG. 5 illustrates a rear perspective view of a prior art syringe encoder including two sets of indicators positioned on different quadrants of the syringe encoder.

FIG. 6A illustrates a side view of a prior art syringe including multiple sets of indicators.

FIG. 6B illustrates a bottom view of the syringe of FIG. 6A.

FIG. 7 illustrates a side cross-sectional view of a prior art syringe encoding system in which energy signals are pulsed.

FIG. 8 illustrates a side cross-sectional view of a prior art syringe encoding system in which syringe configuration is determined in a dynamic fashion.

FIG. 9 illustrates side cross-sectional view of a prior art syringe encoding system using ambient light as a light source for syringe encoding.

FIG. 10A illustrates a side view of a prior art syringe encoding system in which the depth of indicator notches increases with increasing distance from a light source.

FIG. 10B illustrates an expanded view of the encircled area of FIG. 10A.

FIG. 10C illustrates an expanded view of one of the indicator notches of FIGS. 10A and 10B.

FIG. 10D illustrates a prior art indicator notch including an attached reflective surface.

FIG. 11 illustrates a side, cross-sectional view of a prior art syringe encoding system in which indicators redirect energy to one or more sensors positioned within the interior of the syringe.

FIG. 12A is a side view of an embodiment of a syringe encoding system similar to that of FIG. 10A with the addition of an indicator block.

FIG. 12B is a cross-sectional view of an embodiment of a syringe encoding system similar to that of FIG. 10B with the addition of an indicator block.

FIG. 13 is a schematic view of an embodiment of a syringe similar to that of FIG. 1 with the addition of an indicator block.

FIG. 14 is a schematic view of an embodiment of a syringe similar to that of FIG. 3 with the addition of an indicator block.

### DETAILED DESCRIPTION

The encoders, encoding systems, and encoding methods described herein may be particularly useful in encoding information related to configurations for syringes and other pumping mechanisms used in medical injection procedures. Several representative embodiments in which electromagnetic (e.g., light) energy may be used in connection with syringe encoders are discussed below.

In the case that light energy is used, one may, for example, take advantage of the properties of light refraction/reflection at an interface between two different media to assist in efficiently propagating light through the length of the media having the higher refractive index. These different media may, for example, be a translucent or transparent syringe wall and the air surrounding the syringe wall.

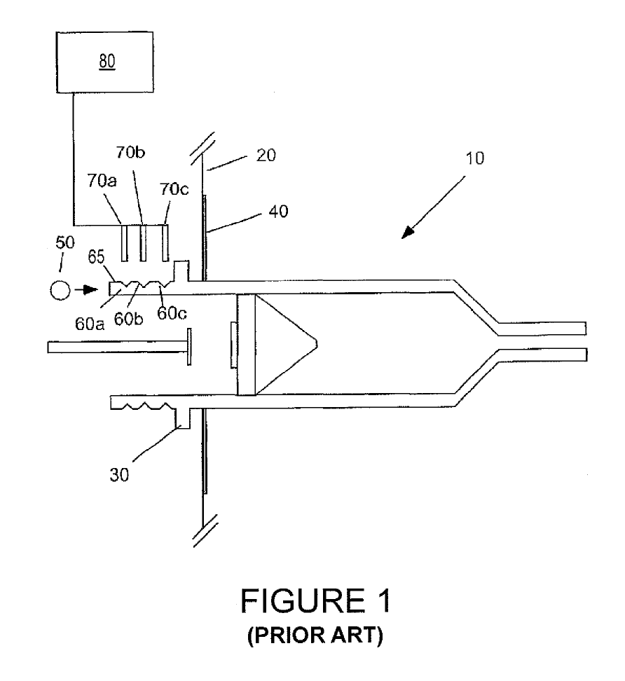

FIG. 1 illustrates a prior art syringe 10 having at least a portion thereof formed from a generally translucent or transparent material such as glass or a clear plastic. Syringe 10 may, for example, be removably positioned upon a powered injector 20 by the interaction of syringe flange(s) 30 and drip flange 40 with mounting means on and/or in the front wall of injector 20. A light source 50 may, for example, be positioned within injector 20 to transmit and/or propagate light energy in a generally axial direction (e.g., parallel to the axis of syringe 10) through a wall 65 of syringe 10. The light energy may be outside the wavelength of visible light to reduce interference from ambient light. Light source 50 may also be pulsed to improve detectability.

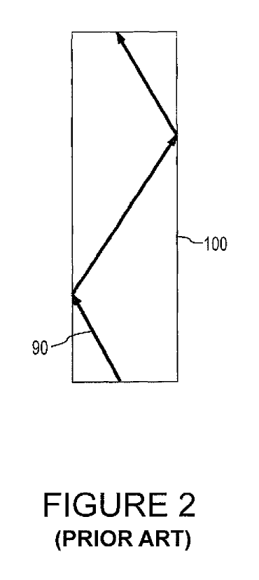

FIG. 2 illustrates light (represented by ray 90) being internally reflected within a prior art syringe wall 100. In general, all light striking the interface between the syringe wall 100 and the air at an angle greater than the critical angle (as measured from a vertical plane in the orientation of FIG. 1 or as measured from a horizontal plane in the orientation of FIG. 2—e.g., a plane normal to the syringe-air interface) may be internally reflected within the syringe wall 100 and propagate therethrough in a generally axial direction.

In one embodiment, syringe 10 may be manufactured from polyethylene terephthalate (PET), for which the index of refraction measured at 632.8 nm (Helium-Neon laser output) is approximately 1.68 for an ambient temperature of 21 degrees C. Given a refractive index of approximately 1.00 for air, this material results in a critical angle for the air-syringe interface of approximately 37 degrees. Therefore, if the light hits the interface at an angle greater than this value, it may be internally reflected. In the case of no scattering or absorption, this reflection is theoretically perfect. Indeed, measurements have shown that the reflection coefficient from a dielectric interface within, for example, a high quality optical fiber exceeds 0.9999. See, for example, Handbook of Optics, McGraw-Hill, p. 13-6. In practice, the reflection coefficient may decrease as imperfections in the material increase.

In FIG. 1, syringe 10 includes a series of indicators 60a-60c that are formed as angled surfaces and/or indicator notches. The indicators 60a-60c act as portals to transmit a portion of the light being propagated through the syringe wall 65 into the surrounding air. Light sensors 70a-70c may be positioned adjacent indicators 60a-60c, respectively, such that each of the light sensors 70a-70c is positioned along a longitudinal axis of the syringe 10 at a point that corresponds to a corresponding one of the indicators 60a-60c. The presence or absence of one or more of indicators 60a-60c (or the relative positions of indicators 60a-60c with respect to each other) may, for example, represent a binary or other code that corresponds to a particular syringe configuration (e.g., a certain volume syringe containing a certain concentration of a particular type of contrast medium) as, for example, interpreted by a processing unit 80 in communicative connection with sensors 70a-70c. Indicators 60a-60c may be placed relatively close to light source 50 to reduce the distance light is transmitted through the syringe wall 65. In this regard, the total light energy available for measurement may decrease as the distance from light source 50 increases (e.g., via scattering, absorption and/or transmission through angled surfaces of indicators 60a-60c). Indicators 60a-60c may be formed around the circumference of syringe 10. In this manner, the orientation of syringe 10 (e.g., the degree of rotation about its axis) is irrelevant to the ability of sensors 70a-70c to measure light transmitted from syringe 10.

Positioning indicators (e.g., indicators 60a-60c of FIG. 1) in general alignment parallel to the axial orientation of syringe 10 and propagating energy from source 50 through the syringe wall 65 generally parallel to the axis of syringe 10, provides substantial space for multiple indicators along the length of syringe 10 and reduces or eliminates problems in propagating energy that may arise from the curvature of the syringe wall 10 around the axis of syringe 10. Moreover, this orientation facilitates positioning of energy source 50 and sensors 70a-70c with only minor changes in existing syringe and injector designs.

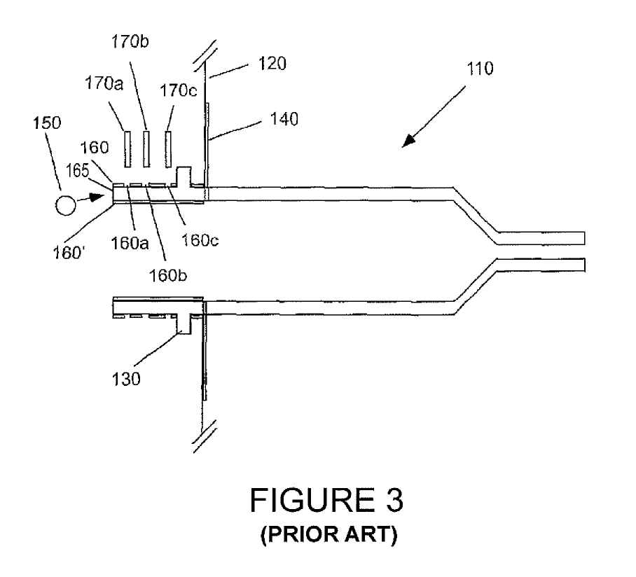

FIG. 3 illustrates an alternative embodiment of a prior art syringe 110 attached to a powered injector 120. As discussed above, syringe 110 includes a mounting flange 130 and a drip flange 140. Injector 120 includes a light source 150 positioned to transmit light into a syringe wall 165 so that light propagates through the syringe wall 165 in a generally axial direction. In this embodiment, at least the rearward section of syringe 110 includes a shield or barrier 160 that may be placed at least on the exterior perimeter of syringe 110. Shield 160 includes several indicators formed as openings or portals 160a-160c that allow light to be transmitted into the surrounding air, whereas the remainder of shield 160 prevents light from being transmitted therethrough. Such light transmitted into the surrounding air may be detected by sensors 170a-170c as discussed above to provide information regarding the syringe configuration. A shield 160′ may also be provided on the interior diameter of the syringe wall 165. Shields 160 and 160′ may, for example, be an opaque plastic and/or an opaque ink. Shields 160 and 160′ may also be reflective to promote the axial propagation of light in an efficient manner.

Although internal reflectance arising from materials of different refractive indices may be useful in efficiently propagating light energy through the length of a medium, internal reflectance may not be necessary. For example, reflective shields or linings as described in connection with FIG. 3 may be used to propagate light energy through a length of material. Moreover, those light rays propagating through a length of material generally parallel to the axis of the length of material (without internal reflection) may interact with indicators in a detectable manner.

In several embodiments, steps may be taken to prevent interference from background or ambient light (e.g., light not originating from the light source(s)). For example, narrow bandwidth detection may be used in which the light source(s) and sensor(s) operate over a very narrow range of optical wavelengths. Moreover, synchronous detection may be used in which the light source(s) may be modulated at some frequency and the sensor electronics may be selectively sensitive to signals varying at that frequency. At the simplest level, the difference in detected signal between a source on state and a source off state may be measured. Many other detection schemes as known, for example, in the optical detection arts may be suitable.

In the embodiments of FIGS. 1 and 3, all indicators (60a-60c and 160a-160c) for directing/transmitting light to sensors (70a-70c and 170a-170c, respectively) are located in or on the syringe wall (65 and 165, respectively), to the rear of drip flanges (40 and 140, respectively). As clear to those skilled in the art, such indicator/sensor pairing may be located anywhere along the syringe wall 65, 165. Moreover, the syringes 10, 110 may include a portion or member that may be separate from the syringe walls 65, 165 through which energy may be transmitted for syringe 10, 110 information encoding.

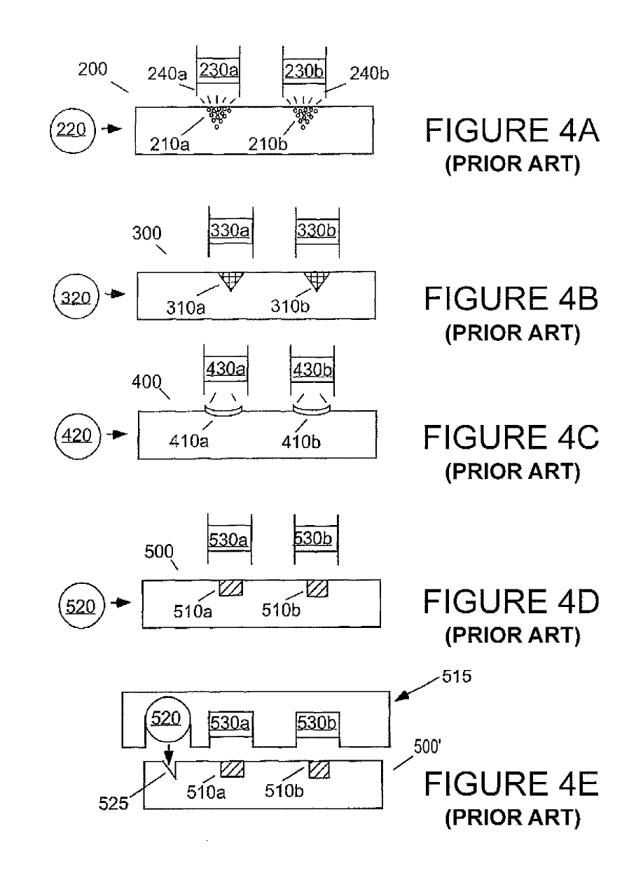

FIGS. 4A through 4E illustrate several further prior art syringe encoder configurations. Each of FIGS. 4A through 4E illustrates a length of material through which electromagnetic energy (e.g., light energy) may pass or propagate. The length of material may, for example, be a portion of a syringe wall, a portion of a syringe adapter or a portion of a syringe or other encoder that is, for example, associated with and/or attachable to syringe, a syringe adapter (e.g., a sleeve that may be positioned adjacent to or that fits over a syringe or a syringe adapter) or another device to be encoded. In general, adapters enable use of syringes not specifically designed for use with a particular injector.

The lengths of material of FIGS. 4A through 4E are referred to simply as syringe encoders below. In FIG. 4A, syringe encoder 200 includes indicators 210a and 210b that may be discontinuities in syringe encoder 200 that act to transmit/redirect/scatter light propagating through syringe encoder 200 from light source 220. Such discontinuities may, for example, be formed as irregularities within the material of syringe encoder 200 or by incorporating another material within syringe encoder 200 (such as by coextrusion of polymeric materials). Light transmitted/redirected/scattered from indicators 210a and 210b may be detected by sensors 230a and 230b, respectively. In the embodiment of FIG. 4A, sensors 230a and 230b may be surrounded by shields or columnators 240a and 240b, respectively. Shields 240a and 240b extend toward the surface of syringe encoder 200 to reduce or prevent light transmitted/redirected/scattered from indicator 220b from being detected by sensor 230a and to prevent light transmitted/redirected/scattered from indicator 220a from being detected by sensor 230b, respectively (sometimes referred to as “crosstalk”). The sensors of FIGS. 4B through 4E may also include such shields.

In FIG. 4B, syringe encoder 300 includes indicators 310a and 310b that absorb light energy propagated through syringe encoder 300 from light source 320 that would otherwise be transmitted outside of syringe encoder 300. Sensors 330a and 330b detect the presence or absence of indicators 310a and 310b as described above. In this embodiment, however, the presence of an indicator at a predetermined position results in the absence of a signal at that position. Whereas if the presence of energy from an indicator is interpreted as a “1” of a binary code, then indicators 210a and 210b of syringe encoder 200 may, for example, correspond to a binary code of 11, indicators 310a and 310b of syringe encoder 300 may correspond to a binary code of 00. It is noted that if the presence of energy from an indicator is interpreted as a “0” of a binary code, then indicators 210a and 210b of syringe encoder 200 may, for example, correspond to a binary code of 00, indicators 310a and 310b of syringe encoder 300 may correspond to a binary code of 11. In general, as discussed herein, the presence of energy from an indicator is interpreted as a “1”, although for any given embodiment, an opposite interpretation may be used.

Syringe encoder 400 of FIG. 4C includes indicators 410a and 410b that act as lenses to focus light being propagated through syringe encoder 400 from light source 420 on sensors 430a and 430b, respectively.

Syringe encoder 500 of FIG. 4D includes indicators 510a and 510b that may be placed in an excited state when light from light source 520 impinges thereupon. For example, indicators 510a and 510b may include a material that fluoresces when light energy impinges thereupon. The excited state (e.g., fluorescence) of indicators 510a and 510b may be detectable by sensors 530a and 530b, respectively. Syringe encoder 500′ of FIG. 4E may be similar in operation to that of syringe encoder 500. However, in the embodiment of syringe encoder 500′, light source 520 is placed in generally the same plane as sensors 530a and 530b. Light from light source 520 may be redirected to propagate through syringe encoder 500′ by angled surface 525. Moreover, in the embodiment of FIG. 4E, light source 520 and sensors 530a and 530b may be incorporated in a carrier 515, which may, for example, be cylindrical sheath such as a syringe heater as known in the art.

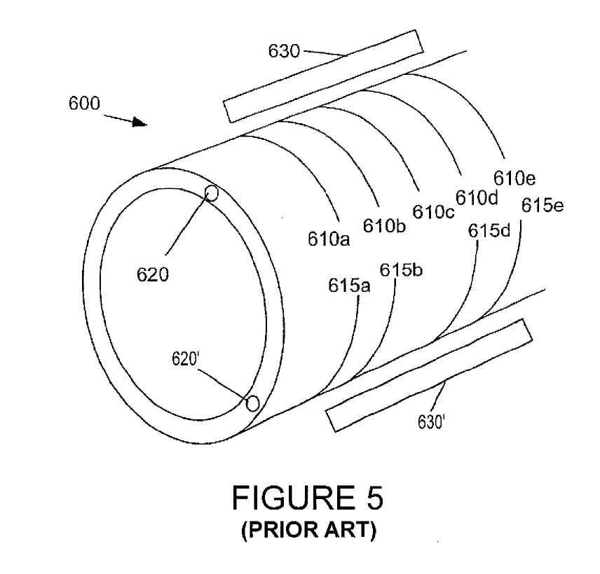

As discussed above, the indicators may, for example, extend around the circumference of a syringe or a syringe adapter to a sufficient extent so that the orientation of the syringe, the syringe adapter, or the syringe encoder (e.g., the degree of rotation about its axis) with respect to the injector, light source and/or sensor bank may be irrelevant to the ability of the corresponding sensors to measure how the indicators modify energy propagated through the syringe, the syringe adapter or the syringe encoder. However, orientation may be used to encode more information. FIG. 5, for example, illustrates a prior art syringe encoder 600 including a plurality (two in this embodiment) of sets of indicators to set forth a plurality of binary codes. Indicators 610a, 610b, 610c, 610d and 610e (the first set) and indicators 615a, 615b, 615d and 615e (the second set) may be positioned, for example, in different sections or quadrants of generally cylindrical syringe encoder 600. Syringe encoder 600 further includes two light sources 620 and 620′ as well as two sensor banks 630 and 630′. Encoder 600 may, for example, be a portion of a syringe wall or a portion of a syringe adapter. Likewise, encoder 600 may be attachable to a syringe or a syringe adapter.

In the embodiment of FIG. 5, at least one indicator in each set of indicators, for example, the last indicator in each set of indicators (e.g., indicators 610e and 615e), may be used to determine if a syringe is properly attached to and/or properly positioned with respect to a powered injector (not shown in FIG. 5). Indicators 610e and 615e (and/or other indicators) may also be used to check parity and/or to calibrate the sensitivity of sensors 630 and 630′, which may, for example, be an array of sensors or a single sensor such as a charge-coupled device (CCD) camera. For example, the indicators of FIG. 5 may be angled notches as discussed in connection with the embodiment of FIG. 1. The amount of light sensed by sensor banks 630 and 630′ as a result of indicators 610e and 615e, respectively, may provide information for calibrating sensitivity settings for determining whether other indicators may be present or absent at various positions on syringe encoder 600.

Dedicating the use of indicators 610e and 615e as position and/or calibration indicators, the presence or absence of other indicators may be used to set forth binary code(s) of predetermined lengths. In FIG. 5, two binary codes of four bits each are represented by indicators 610a, 610b, 610c and 610d of the first set of indicators and by indicators 615a, 615b and 615d of the second set of indicators. The binary code of the first set of indicators is 1111, while the binary code of the second set of indicators is 1101 (an indicator at the third or “c” position is absent in the second set of indicators). The two binary codes correspond to a particular syringe configuration as may be provided, for example, in a look up table stored in computer memory. With the use of a sensor or sensors having a relatively wide detection range (e.g., a CCD camera), the absolute position of a set of indicators representing a binary code may not be as important as the case in which sensors having a relatively narrow range of detection are used, requiring general alignment of an indicator/sensor pairing.

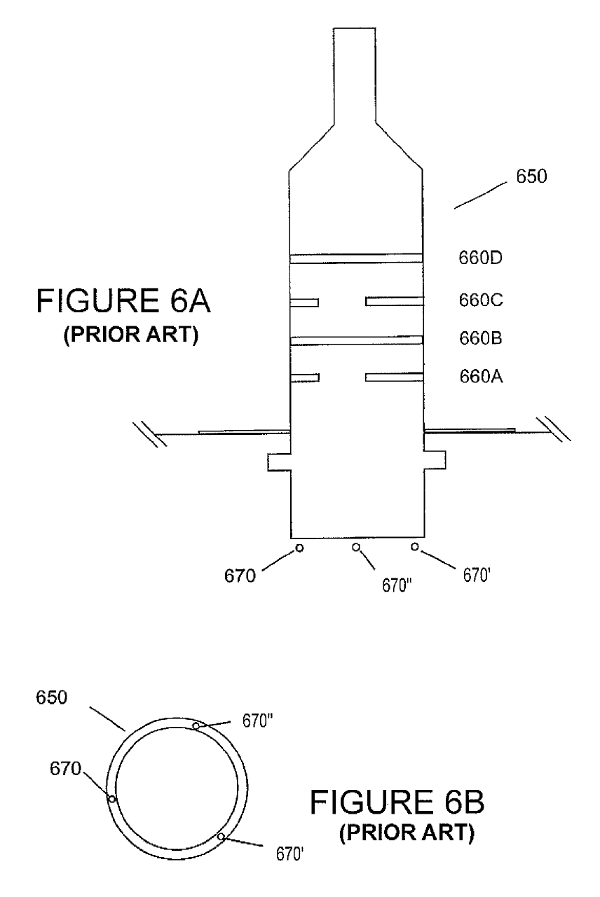

FIGS. 6A and 6B illustrate another prior art configuration (similar to that of FIG. 5) in which several bands of indicators 660A, 660B, 660C and 660D extend at least partially around the circumference of a syringe 650 at predetermined positions along the length of syringe 650. As illustrated in FIG. 6B, three energy sources 670, 670′ and 670″ may be positioned at different positions around the circumference of syringe 650 adjacent the rearward end of syringe 650. Four detectors (not shown in FIGS. 6A and 6B) may be placed in general alignment with sources 670, 670′ and 670″ at each band level of indicators (four bands X three sources=twelve detectors in total). Dedicating, for example, the D-band of indicators to position and/or calibration determinations as described above, one is left with three binary codes of three bits each or 512 possible different encoded configurations.

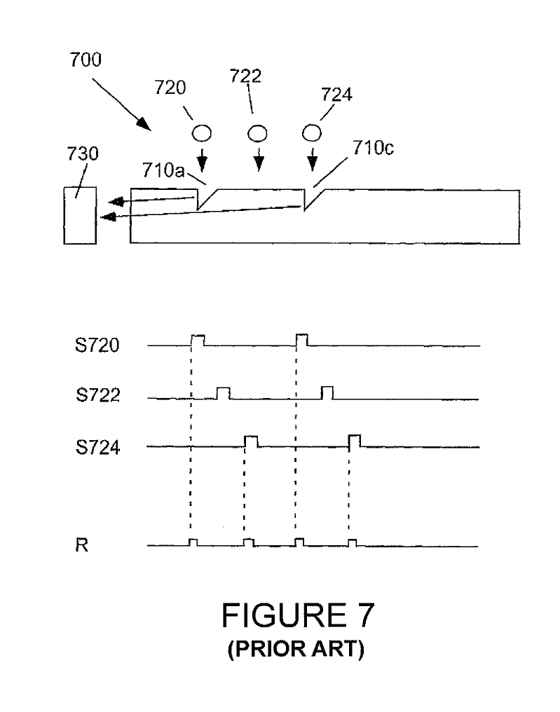

In FIG. 7, a prior art syringe encoder 700 includes indicators 710a and 710c that may be angled surfaces formed in the surface of syringe encoder 700. Three energy sources 720, 722, 724 may be pulsed sequentially as shown in the timing diagram of FIG. 7 as waveforms S720, S722, S724. Energy sources 720 and 724 may be positioned over indicators or grooves 710a and 710c, respectively, in the syringe barrel, which transmit light to a receiver 730. In the embodiment of FIG. 7, there is no indicator on the syringe corresponding to the fixed position of energy source 722. No energy may, therefore, be transmitted to receiver 730 when waveform S722 is pulsed on. Consequently, the reception portion R of the timing diagram shows pulses received from S720 and S724 but not from S722. The presence or absence of indicators at each source may represent a digital code as described above.

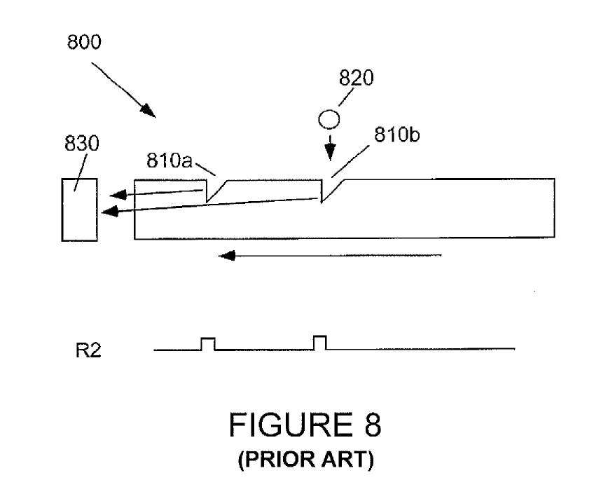

In the above discussion, syringe configuration information may be read in a static fashion. Syringe configuration information may also be read in a dynamic fashion. As prior art syringe encoder 800 is moved to the left in the orientation of FIG. 8 (e.g., as a syringe is attached to a powered injector), indicators 810a and 810b redirect at least a portion of light energy from light source 820 through syringe encoder 800 to a receiver 830 as illustrated with arrows in FIG. 8. A received signal R2 provides information on syringe configuration.

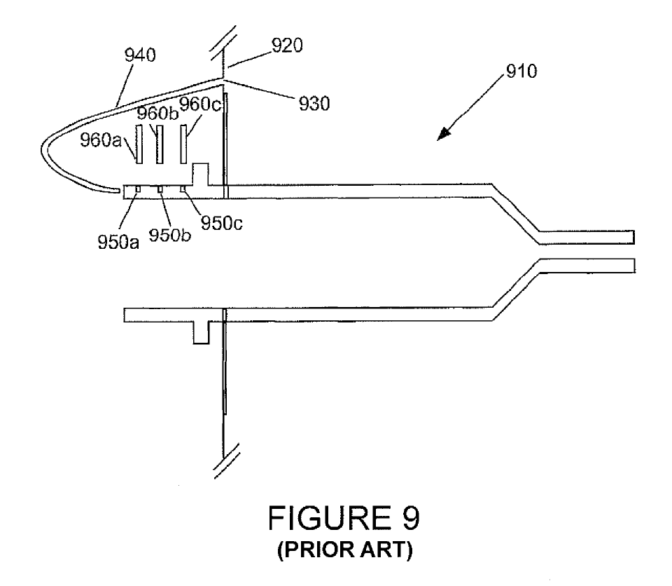

In the case that light energy is used, the light source may be a powered light source such as an LED and/or other powered light source as know in the art. However, ambient light may also be used. In FIG. 9, for example, a prior art syringe 910 is attached to a powered injector 920. Powered injector 920 includes an opening 930 through which ambient light may pass. Opening 930 may be in communicative connection with, for example, a fiber optic cable 940. Fiber optic cable 940 terminates adjacent a rearward end of syringe 910 and provides light energy to one or more indicators 950a, 950b and 950c. As discussed above, detectors 960a, 960b and 960c may be adapted to sense modification of the light energy by indicators 950a, 950b and 950c, respectively.

Light transmitted to a sensor (as measured, for example, in brightness or signal strength) may be sufficient such that the interaction of light with an indicator may be readily detectable using commercially available, inexpensive sensors and light sources. An example of a suitable sensor is the SFH229FA (part number) photodiode produced by OSRAM, a multinational corporation headquartered in Munich, Germany. An example of a suitable light source is the HSDL-4230 (part number) LED produced by Hewlett-Packard, a multinational corporation headquartered in Palo Alto, Calif.

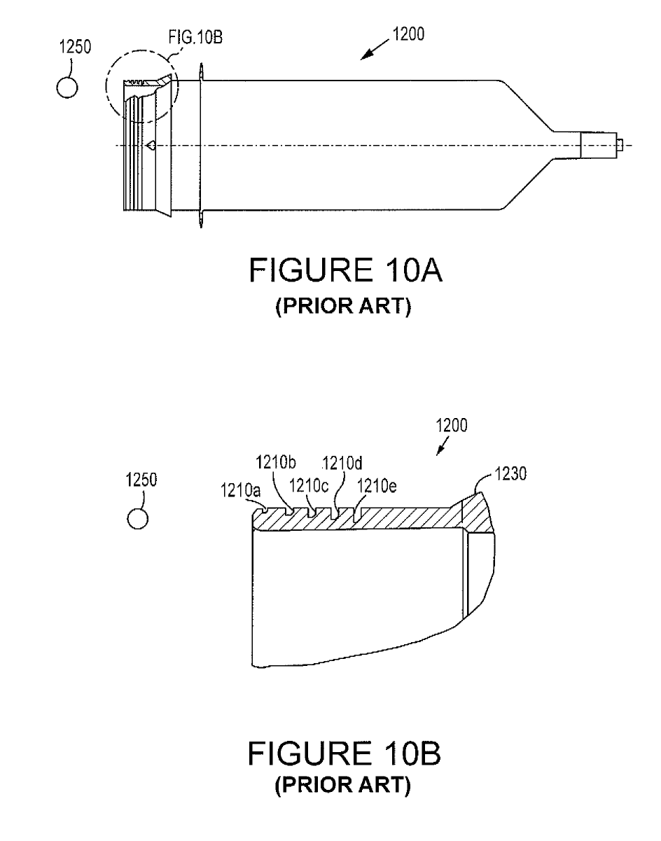

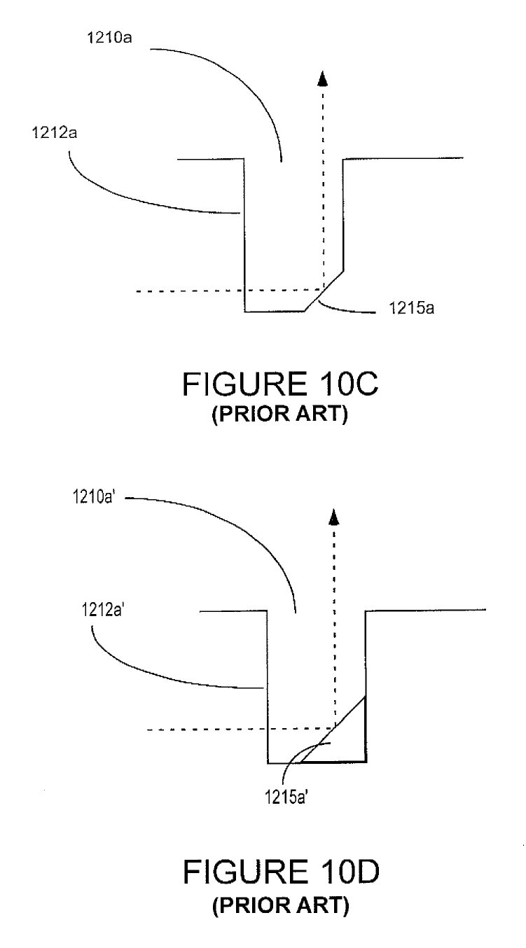

FIGS. 10A through 10D illustrate a prior art syringe 1200 in which indicator notches 1210a through 1210e increase in depth with increasing distance from a light source 1250. FIG. 10B illustrates an expanded view of indicator notches 1210a through 1210e (e.g., the encircled portion of FIG. 10A). Indicator notches 1210a through 1210e may be placed at a rearward position on syringe 1200 to position indicator notches 1210a through 1210e as close as possible to the light source as well as to reduce or prevent undesirable signal artifacts arising from other syringe components. Placing indicator notches 1210a through 1210e between the energy/light source and such syringe components reduces the likelihood of undesirable signal artifacts.

FIG. 10C illustrates an expanded view of indicator notch 1210a of FIGS. 10A and 10B. As illustrated in FIG. 10C, a light ray first passes through a generally perpendicular wall 1212a of indicator notch 1210a and then passes through the air to impinge upon surface 1215a, which reflects the light ray upward to a sensor (not shown in FIG. 10C). Surface 1215a in FIG. 10C is a portion of the syringe wall angled at an approximately 45 degree angle to light rays propagating lengthwise through the wall of syringe 1200. FIG. 10D illustrates another embodiment of an indicator notch 1210a′. In the embodiment of FIG. 10D, a light ray first passes through a generally perpendicular wall 1212a′ of indicator notch 1210a′ and then passes through the air to impinge upon surface 1215a′, which reflects the light ray upward to a sensor (not shown in FIG. 10D). In the embodiment of FIG. 10D, reflective surface 1215a′ may be formed of a different material (e.g., a highly reflective material) than the material of syringe 1200.

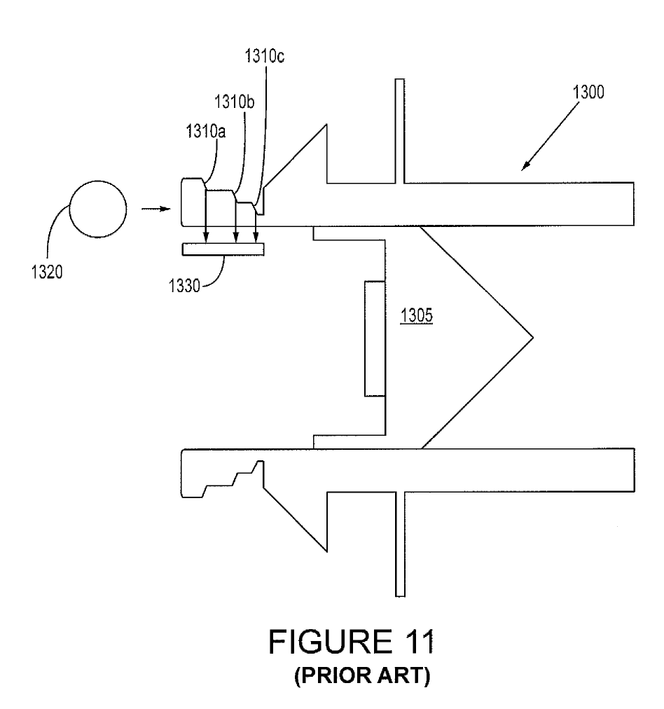

FIG. 11 illustrates a rear portion of a prior art syringe 1300 including indicators 1310a-1310c formed as angled steps in the exterior wall of syringe 1300. In one embodiment, indicators 1310a-1310c may be angled at approximately 45 degrees with respect to light rays propagated through the wall of syringe 1300 from light source 1320. In this embodiment, light rays from light source 1320 may be reflected at an angle of approximately 90 degrees with respect to the orientation through which the light is propagated through the wall of syringe 1300 toward a sensor or sensors 1330 positioned on the interior side of the syringe wall. Reflection of light at generally right angles may facilitate positioning of a corresponding sensor or sensors for detection of reflected light. In this embodiment, indicators 1310a-1310c affect the light energy generally independently of each other. Sensor or sensors 1330 may be positioned within the interior of the barrel of syringe 1300 to minimize or prevent interference with the movement of a plunger 1305 within the syringe barrel.

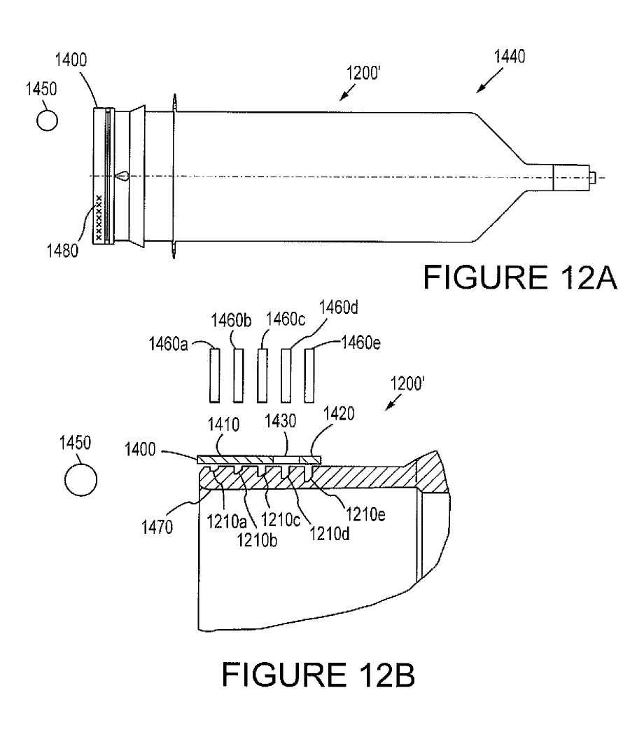

FIG. 12A is a side view of an embodiment of a syringe 1200′ similar to that of syringe 1200 of FIG. 10A with the addition of an indicator block 1400. FIG. 12B is a cross-sectional view of a portion of the syringe 1200′ and the indicator block 1400 similar in orientation to FIG. 10B. Together, the syringe 1200′ and indicator block 1400 form a syringe assembly 1440. The indicator block 1400, in conjunction with the syringe 1200′, may be operable to encode the syringe assembly 1440 in a manner so that it may be read in a way similar to syringe 1200 of FIG. 10A and/or other previously discussed indicator/syringe combinations. In this regard, the syringe 1200′ may include indicators or optical encoding elements 1210a-1210e at every potential location (e.g., all five possible indicator locations of syringe 1200).

Each indicator 1210a-1210e may be operable to redirect electromagnetic energy propagated through a wall 1470 of syringe 1200′ from source 1450. The wall 1470 may be in the form of a wall 1470 of the syringe 1200′ or may be a length of material as described above. This would be the equivalent of the syringe 1200 of FIG. 10A encoded with 11111 (all indicators 1210a-1210e present). In this regard, energy from the source 1450 may be reflected at an angle of approximately 90 degrees (with respect to the orientation through which the energy is propagated through the wall 1470 of syringe 1200′) toward corresponding sensors 1460a-1460e positioned outside the wall 1470 of the syringe 1200′. However, this energy may be selectively blocked. For example, the indicator block 1400 of FIGS. 12A and 12B includes opaque portions 1410, 1420 and transparent (e.g., transparent to the electromagnetic energy emitted by source 1450) portion 1430. The opaque portions 1410, 1420 are positioned between indicators 1210a, 1210b, 1210c and 1210e and their corresponding sensors 1460a, 1460b, 1460c and 1460e, respectively. The transparent portion 1430 is positioned between indicator 1210d and its corresponding sensor 1460d. The result of the configuration of the indicator block 1400 is that only energy reflected by indicator 1210d is able to reach its corresponding sensor 1460d, resulting in a binary code reading of 00010. In this regard, 0 represents a reduced level of energy from the source 1450 reaching the sensor, while 1 represents a greater level of energy from the source 1450 reaching the sensor. In this context, “reduced level” and “greater level” are relative to each other and represent a difference between them that is discernable by the sensors 1460a-1460e.

Any binary code ranging from 00000 (a completely opaque indicator block 1400) to 11111 (a completely transparent indicator block 1400) may be achieved by an appropriately configured indicator block 1400 and the syringe 1200′ that includes indicators 1210a-1210e at every potential location. That is, by appropriately placing opaque portions or transparent portions between appropriate indicators 1210a-1210e and their corresponding sensors 1460a-1460e, respectively, any binary code from 00000 to 11111 may be achieved. Moreover, such syringe assemblies 1440 may be substituted for syringe 1200 for use in the power injectors described herein.

The indicator block 1400 may encircle the entire syringe 1200′ such that regardless of the orientation of the syringe assembly 1440 in the power injector, the sensors 1460a-1460e will be able to correctly read the binary code of the syringe assembly 1440.

Where the electromagnetic energy from the source 1450 is visible light, the transparent portion 1403 may be clear and the opaque portions 1410, 1420 may be opaque to visible light. In an embodiment, the transparent portion 1430 may be replaced by the absence of material. For example, in such an embodiment, the indicator block 1400 of FIG. 12B may include a first portion that blocks energy from indicators 1210a-1210c, and a second portion that blocks energy from indicator 1210d. The two portions may be connected (e.g., by this strips thin enough that the would not interfere with the operation of the sensor 1460d if they were directly between the sensor 1460d and the indicator 1210d) or unconnected (e.g., two separate indicator blocks that may be installed independent of each other).

The indicator block 1400 may be in the form of a label (e.g., an adhesive backed label) that may be installed onto the syringe 1200′ by wrapping the label around the syringe 1200′. In such an embodiment, the label may be sized and/or configured in such a way as to aid in the manual installation and/or inspection of the label. For example, the label may be configured as shown in FIG. 12B such that upon installation, an edge of the label is aligned with an edge of the syringe 1200′ (e.g., the rear edge of the syringe 1200′ proximate to indicator 1210a). Such a configuration may assist in the manual installation of the indicator block 1400. In another embodiment, the indicator block 1400 in the form of a label may be configured for automated installation onto the syringe 1200′.

The indicator block 1400 may be in any other appropriate form. For example, the indicator block 1400 may be an elastic band that may be operable to fit over the syringe 1200′. In another example, the indicator block 1400 may be operable to press fit onto the syringe 1200′. In yet another example, the indicator block 1400 may be in the form of ink, paint, or the like, that is applied over the appropriate indicators 1210a-1210e.

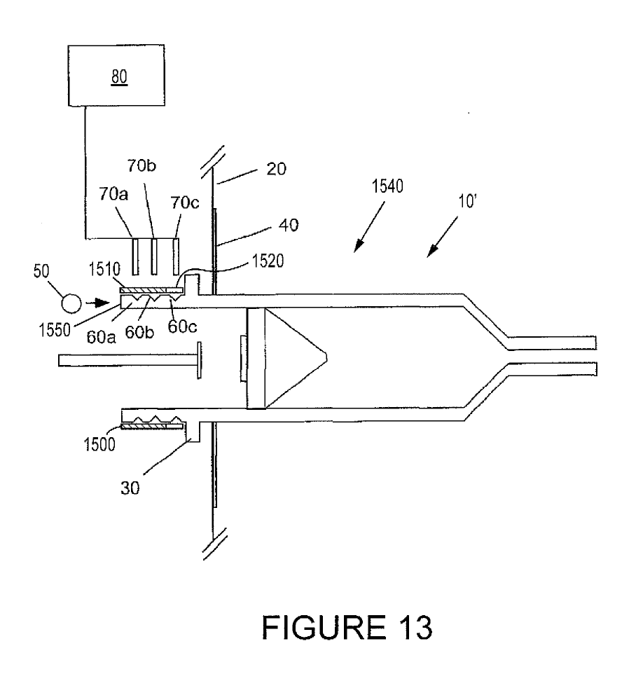

FIG. 13 is a cross-sectional view of an embodiment of a syringe 10′ with the addition of an indicator block 1500. Together, the syringe 10′ and indicator block 1500 form a syringe assembly 1540. The indicator block 1500, in conjunction with the syringe 10′, may be operable to encode the syringe assembly 1540 in a manner so that it may be read in a way similar to syringe 10 of FIG. 1 and/or other previously discussed indicator/syringe combinations. In this regard, the syringe 10′ may include indicators or optical encoding elements 60a-60c at every potential location (e.g., all three possible indicator locations of syringe 10′). Accordingly, each indicator 60a-60c may be operable to redirect electromagnetic energy propagated through a wall 1550 of syringe 10′ from source 50. The wall 1550 may be in the form of a wall 1550 of the syringe 10′ or may be a length of material as described above.

However, this energy may be selectively blocked. For example, the indicator block 1500 of FIG. 13 includes an opaque portion 1510 and transparent portion 1520. The opaque portion 1510 is positioned between indicators 60a and 60b, and their corresponding sensors 70a and 70b, respectively. The transparent portion 1520 is positioned between indicator 60c and its corresponding sensor 70c. The result of the configuration of the indicator block 1510 is that only energy reflected by indicator 60c is able to reach its corresponding sensor 70c, resulting in a binary code reading of 001. The opaque portion 1510 and transparent portion 1520 may be configured similar to the opaque portions 1410, 1420 and transparent portion 1430 of indicator block 1400, respectively.

Any binary code ranging from 000 (a completely opaque indicator block 1500) to 111 (a completely transparent indicator block 1500) may be achieved by an appropriately configured indicator block 1500 and the syringe 10′ that includes indicators 60a-60c at every potential location. Moreover, such syringe assemblies 1540 may be substituted for syringe 10 for use in the power injector 20. Furthermore, the indicator block 1500 may be configured for attachment to the syringe 10′ in any appropriate manner, such as those discussed above with reference to indicator block 1400.

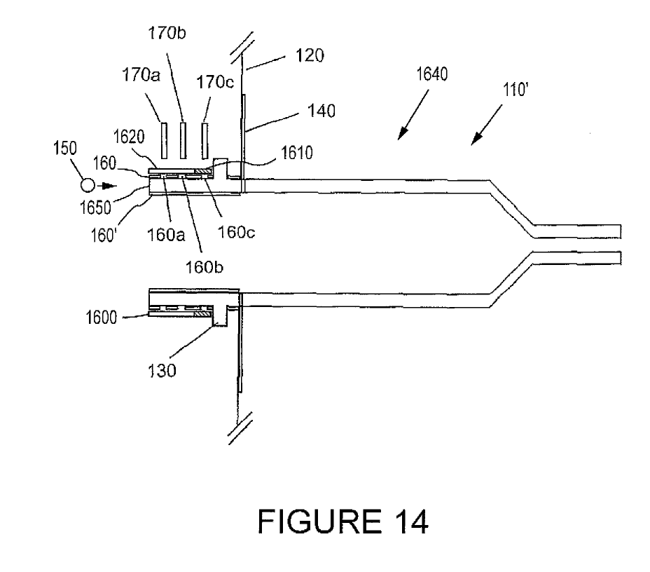

FIG. 14 is a cross-sectional view of an embodiment of a syringe 110′ with the addition of an indicator block 1600. Together, the syringe 110′ and indicator block 1600 form a syringe assembly 1640. The indicator block 1600, in conjunction with the syringe 110′, may be operable to encode the syringe assembly 1640 in a manner so that it may be read in a way similar to syringe 110 of FIG. 3 and/or other previously discussed indicator/syringe combinations. In this regard, the syringe 110′ may include indicators or optical encoding elements 160a-160c at every potential location (e.g., all three possible indicator locations of syringe 110′). Accordingly, each indicator 160a-160c may be operable to redirect electromagnetic energy propagated through a wall 1650 of syringe 110′ from source 150. The wall 1650 may be in the form of a wall 1650 of the syringe 110′ or may be a length of material as described above.

However, this energy may be selectively blocked. For example, the indicator block 1600 of FIG. 14 includes an opaque portion 1610 and transparent portion 1620. The opaque portion 1610 is positioned between indicator 160c, and its corresponding sensor 170c. The transparent portion 1620 is positioned between indicators 160a and 160b and their corresponding sensors 170a and 170b, respectively. The result of the configuration of the illustrated indicator block 1600 is that only energy reflected by indicators 160a and 160b is able to reach the corresponding sensors 170a and 170b, resulting in a binary code reading of 110. The opaque portion 1610 and transparent portion 1620 may be configured similar to the opaque portions 1410, 1420 and transparent portion 1430 of indicator block 1400, respectively.

Any binary code ranging from 000 (a completely opaque indicator block 1600) to 111 (a completely transparent indicator block 1600) may be achieved by an appropriately configured indicator block 1600 and the syringe 110′ that includes indicators 160a-160c at every potential location. Moreover, such syringe assemblies 1640 may be substituted for syringe 110 for use in the power injector 120. Furthermore, the indicator block 1600 may be configured for attachment to the syringe 110′ in any appropriate manner, such as those discussed above with reference to indicator block 1400.

In general, indicator blocks may be configured to work with any of the syringe embodiments discussed to herein, where the syringe contains indicators at every potential location. Thus, for such syringes, the binary encoding will result from the configuration of an appropriate indicator block. One advantage of such indicator blocks is that the syringes to be used in the power injectors may all be identically configured (e.g., with all potential indicators present), and thus only one type of syringe need be manufactured and kept in inventory. Uniquely encoded syringe assemblies may be achieved by applying appropriate indicator blocks to the syringes. Accordingly, inventory may consist the standard type of syringe (e.g., with all potential indicators present) and a variety of indicator blocks. This may be a lower cost (e.g., lower carrying costs for inventory) system than a system where a variety of uniquely manufactured syringes (e.g., syringes encoded during the manufacturing process by the inclusion/deletion of various indicators) must be kept in inventory.

Another characterization of the syringe assemblies described above in relation to FIGS. 12A-14 is that a single syringe configuration (e.g., a generic syringe configuration) may be manufactured with a plurality of indicators or optical encoding elements. Each of a plurality of different combinations of one or more indicators/optical encoding elements may define an indicator or encoding set. Each indicator/encoding set may correspond with what may be characterized as an information set, data set, or encoded information that differs in at least some respect from every other information set. The various indictor/encoding sets may be defined by selectively applying one or more indicator blocks to a syringe of the generic syringe configuration. Each indicator block may be of any appropriate size, shape, configuration and/or type, and furthermore may be applied to (e.g., mounted) to a syringe of the generic syringe configuration in any appropriate manner.

An indicator or encoding set may be defined by mounting at least one indicator block on the syringe such that it blocks transmission of an optical signal from at least one of the optical encoding elements. Each indicator/encoding set may thereby be in the form of a binary code—for example, a “1” for the case where the optical signal from a particular optical encoding element is able to progress to its corresponding optical detector or sensor and a “0” for the case where the optical signal from a particular optical encoding element is blocked by an indicator block such that this optical signal does not reach its corresponding optical detector or sensor.

The syringe assemblies described in relation to FIGS. 12A-14 may be in the form of pre-filled syringes. A “pre-filled syringe,” as used herein, means that the syringe is loaded with medical fluid at a first location (e.g., a production facility) and is transported (e.g., in bulk with other pre-filled syringes) to a second location (e.g., an end use facility) in a common shipping container with other pre-filled syringes. In this regard, the fluid is loaded into the syringe and at least one indicator block is mounted on the syringe before shipping the pre-filled syringe in accordance with the foregoing.

The indicator blocks 1400, 1500, and 1600 described herein have been described in conjunction with selected syringes 1200′, 10′ and 110′, respectively. It should be noted that appropriately configured indicator blocks may be used with any of the syringes described herein. Furthermore, indicator blocks may be used with other syringe configurations that include indicators in every potential location. Such syringe configurations may include any appropriate total number of potential indicator locations for encoding any appropriate length binary code. Indicator blocks may be operable to work in encoding systems, such as the syringe encoder 600 (FIG. 5), where multiple binary codes are represented by multiple sets of indicators disposed at various positions about the circumference of a syringe. Such indicator blocks may be installed in a particular orientation relative the syringe to ensure proper alignment of the transparent and/or opaque sections with the circumferentially positioned indicators.

The indicator blocks described herein may also contain additional information in the form of printed matter. For example, human-readable text (e.g., indicia 1480 in FIG. 12A) may be printed onto opaque portions of the indicator blocks to provide additional identification capability. Barcodes and/or other machine-readable items may be placed onto the indicator blocks. Such additional information may be beneficial for inventory tracking or any other circumstance where it may be beneficial to identify the syringe assembly away from the power injector and/or other devices with the capability to read the binary code encoded in the indicator block.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

What is claimed:

1. A syringe assembly for use with an injector having a plurality of sensors located at different predetermined longitudinal positions on said injector, said syringe assembly comprising:
a body comprising a wall and defining a longitudinal syringe axis;
an mounting mechanism to enable said syringe assembly to be mounted to said injector;
a length of material disposed along at least a portion of said wall, said length of material adapted to propagate electromagnetic energy therethrough in a direction substantially parallel to said longitudinal syringe axis, said length of material comprising at least two indicators, each of said indicators being located at a different predetermined longitudinal position along said length of material, each of said indicators being positioned to align with a corresponding sensor when said syringe assembly is attached to said injector, each of said indicators being adapted to interact concurrently with at least a portion of said energy being propagated through said length of material in a direction substantially parallel to said longitudinal syringe axis in a manner that is readily detectable by said corresponding sensor; and
an indicator block disposed to block the propagation of electromagnetic energy from at least one of said at least two indicators to a corresponding sensor, said at least two indicators and indicator block providing information about said syringe assembly configuration in the form of a binary code on the basis of presence or absence of electromagnetic energy from one or more of said indicators at predetermined longitudinal positions along said length of material reaching corresponding sensors.

2. A syringe assembly comprising:
a body defining a longitudinal syringe axis;
a plunger movably disposed within said body;
a length of material disposed along at least a portion of said body and being adapted to propagate light energy therethrough in a direction substantially parallel to said longitudinal syringe axis, said length of material comprising at least two indicators located at unique predetermined positions therealong, each of said indicators being adapted to redirect at least a portion of said light energy outside of said body in a manner that is detectable, each of said indicators being positioned at a different depth within said length of material;
an indicator block disposed to block the propagation of light energy from at least one of said at least two indicators to a corresponding sensor, said light redirected from said indicators, and not blocked by said indicator block, providing a code that provides information about said syringe assembly configuration; and
at least one mounting flange associated with said body.

3. A syringe assembly comprising a body comprising a wall and defining a longitudinal syringe axis, a length of said wall being adapted to propagate electromagnetic energy therethrough in a direction generally parallel to said longitudinal syringe axis, said wall comprising at least two indicators positioned at unique and different predetermined longitudinal positions therealong, each of said indicators being positioned at a different depth within said wall so that each of said indicators are adapted to interact concurrently with at least a portion of the electromagnetic energy being propagated through said wall to redirect light outside of said wall in a manner that is detectable, an indicator block disposed to block the propagation of electromagnetic energy from at least one of said at least two indicators to a corresponding sensor, the electromagnetic energy redirected from said indicators, and not blocked by said indicator block, providing a code that provides information about said syringe assembly configuration.

4. The syringe assembly of claim 1, wherein the total number of indicators of said syringe assembly is equal to the total number of sensors of said injector.

5. The syringe assembly of claim 1, wherein each consecutive pair of said at least two indicators is separated by an intermediate region of said length of material, wherein each of said intermediate regions comprises an opaque portion that prevents the energy being propagated through said length of material from leaving said length of material in a direction away from said syringe assembly and perpendicular to said longitudinal syringe axis.

6. The syringe assembly of claim 1, wherein each consecutive pair of said at least two indicators is separated by an intermediate region of said length of material, wherein each of said intermediate regions is free from a feature designed to redirect said energy away from a direction substantially parallel to said longitudinal syringe axis.

7. The syringe assembly of claim 3, wherein each consecutive pair of said at least two indicators is separated by an intermediate region of said wall, wherein each of said intermediate regions comprises an opaque portion that prevents the energy being propagated through said wall from leaving said wall in a direction away from said syringe assembly.

8. The syringe assembly of claim 3, wherein each consecutive pair of said at least two indicators is separated by an intermediate region of said wall, wherein each of said intermediate regions is free from a feature designed to redirect said energy away from a direction substantially parallel to said longitudinal syringe axis.

9. The syringe assembly of claim 1, wherein said syringe assembly comprises five indicators.

10. The syringe assembly of claim 1, wherein said indicator block is in the form of a label.

11. The syringe assembly of claim 1, wherein said indicator block is adhesive-backed.

12. The syringe assembly of claim 1, wherein said indicator block comprises indicia related to contents of said syringe assembly.

13. The syringe assembly of claim 1, wherein said indicator block comprises at least one opaque region disposed between one of said indicators and its corresponding sensor.

14. The syringe assembly of claim 1, wherein said indicator block comprises at least one transparent region disposed between one of said indicators and its corresponding sensor and at least one opaque region disposed between another one of said indicators and its corresponding sensor.

15. The syringe assembly of claim 1, wherein said indicator block encircles an entirety of said syringe assembly.

16. A method of encoding a syringe for automated identification of said syringe, said method comprising:
filling said syringe with a predetermined medical fluid type, wherein said syringe comprises a body comprising a wall and defining a longitudinal syringe axis;
selecting a label corresponding to said predetermined medical fluid type, wherein said selected label comprises an opaque region; and
applying said selected label to said syringe such that said opaque region is disposed over a first indicator of said syringe, while at least a second indicator of said syringe is free from having said opaque region disposed thereover, wherein said first and second indicators are adapted to interact concurrently with at least a portion of energy propagated through a length of said syringe in a direction substantially parallel to said longitudinal syringe axis in a manner that is readily detectable by corresponding sensors in longitudinal alignment with said first and second indicators.

17. The method of claim 16, wherein said applying step further comprises applying said selected label such that a transparent region of said selected label is disposed over said second indicator.

18. The method of claim 16, wherein said applying step comprises:
peeling a disposable backing away from said label to expose adhesive disposed on a back side of said label;
aligning one of said opaque regions with said first indicator; and
contacting said back side of said label to said syringe after said aligning and peeling steps.

19. A method of providing medical fluid comprising the method of claim 16 and shipping said syringe after said filling and applying steps, wherein during said shipping said syringe comprises a pre-filled syringe.

20. A syringe assembly comprising:
a body comprising a plurality of optical encoding elements adapted to transmit an optical signal;
a plunger comprising a plunger head movably disposed within said body; and
an indicator block separately mounted on said body in position to block transmission of an optical signal from at least one of said optical encoding elements.

21. The syringe assembly of claim 20, wherein said body comprises a syringe barrel.

22. The syringe assembly of claim 20, wherein said plurality of optical encoding elements are spaced along a longitudinal axis along which said plunger moves relative to said body.

23. The syringe assembly of claim 20, wherein a first encoding set corresponds to first encoded information, wherein a second encoding set corresponds to second encoded information, wherein said first and second encoding sets each comprise at least one optical encoding element of said plurality of optical encoding elements having an optical signal that fails to be blocked by said indicator block, and wherein said first and second encoding sets are different.

24. The syringe assembly of claim 20, wherein a first encoding set comprises a first combination of at least some of said plurality of optical encoding elements and corresponds with first encoded information, wherein a second encoding set comprises a second combination of at least some of said plurality of optical encoding elements and corresponds with second encoded information, and wherein said first and second combinations are different.

25. The syringe assembly of claim 23, wherein said first encoded information differs from said second encoded information.

26. The syringe assembly of claim 20, further comprising fluid in said body prior to installing said syringe assembly on an injector.

27. The syringe assembly of claim 20, wherein said syringe assembly comprises a prefilled syringe.

28. The syringe assembly of claim 20, wherein said indicator block is in the form of a label.

29. The syringe assembly of claim 20, wherein said indicator block is adhesive-backed.

30. The syringe assembly of claim 20, wherein said indicator block comprises indicia related to contents of said syringe assembly.

31. The syringe of claim 20, wherein said indicator block comprises at least one transparent region corresponding to at least one of said plurality of optical encoding elements.

32. The syringe assembly of claim 20, wherein said indicator block encircles an entirety of said syringe assembly.
